(19) United States
(12) Patent Application Publication
(10) Pub. No.: US 2021/0052767 A1
(43) Pub. Date: Feb. 25, 2021

(54) ANTIMICROBIAL FIBER COMPRISING SILVER, FABRIC AND WOUND DRESSING COMPRISING THE ANTIMICROBIAL FIBER, AND METHODS FOR MANUFACTURING THE FIBER, THE FABRIC, AND THE WOUND DRESSING

(71) Applicant: Foshan United Medical Technologies Ltd., Foshan (CN)

(72) Inventors: Xiaodong WANG, Foshan (CN); Bingzhi TAO, Foshan (CN); Xiaohui MO, Foshan (CN); Haitao XU, Foshan (CN)

(21) Appl. No.: 17/090,954

(22) Filed: Nov. 6, 2020

Related U.S. Application Data
(63) Continuation-in-part of application No. 14/437,220, filed on Apr. 21, 2015.

(30) Foreign Application Priority Data
Apr. 23, 2012 (CN) 201210122966.3

(51) Int. Cl. A61L 15/46; A61L 15/28; A61F 13/00; B65B 5/04; B65B 55/12; D01F 1/10; D01F 9/04

(52) U.S. Cl. CPC A61L 15/46; A61L 15/28; A61F 13/00063; A61F 13/00991; A61L 2300/104; B65B 55/12; D01F 1/103; D01F 9/04; B65B 5/04

(57) ABSTRACT

A wound dressing, including fibers containing silver ions. The fibers are manufactured by dissolving silver nitrate into a polymer solution in the substantial absence of light, and then extruding the obtained solution into fibers by wet spinning process. The silver content of the wound dressing is between 0.01-10% by weight, and preferably 0.1-7% by weight.

### CROSS-REFERENCE TO RELATED APPLICATIONS

This application is a continuation-in-part of and claims domestic priority benefits to U.S. application Ser. No. 14/437,220, filed on Apr. 21, 2015, now pending, which is a National Stage Appl. filed under 35 U.S.C 371 of International Patent Application No. PCT/CN2013/074345 with an international filing date of Apr. 18, 2013, designating the United States, and further claims foreign priority benefits to Chinese Patent Application No. 201210122966.3 filed Apr. 23, 2012. The contents of all of the aforementioned applications, including any intervening amendments thereto are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, and Cambridge, Mass. 02142.

### BACKGROUND

This invention relates to an antimicrobial fiber comprising silver, to fabric and wound dressing comprising the antimicrobial fiber, and to methods for manufacturing the fiber, the fabric, and the wound dressing. The wound dressing functions to release silver ions continuously to a wound site, providing an effective antibacterial function and preventing wound infections. The wound dressing is suitable for the management of chronic wounds.

Conventional antimicrobial silver wound dressings can be divided into two types: metallic silver dressing and ionic silver dressing. The metallic silver dressing is conventionally manufactured by coating silver onto the fiber surface or by adding silver metal such as nano silver into the fiber structure, while ionic silver dressings are conventionally manufactured by adding silver compounds into the fiber structure. Both wound dressings can release silver ions to kill microorganisms when in contact with wound.

Ionic silver wound dressing has a high utilization efficiency of silver and is widely used in the wound dressing industry. However, the ionic silver compound is also associated with low solubility. Materials such as silver chloride and silver carbonate have low solubility in water, often resulting in a very small amount of silver ions being released before reaching equilibrium. Only when these sliver ions are depleted can more silver ions be released again. This release-consumption-release cycle can provide a continuous release of silver ions, but, because of the low solubility, the amount of available silver ions in the water contacting with the wound dressing is always small. Therefore, relatively large loading of silver-containing materials is needed in order to achieve a desired antimicrobial performance.

### SUMMARY

In order to address the above issues, this invention provides a method to add the silver ionic directly into the spinning solution by utilizing the bonding of the —COOH and —NH₂ in the spinning solution of alginate and chitosan to silver ions. By adding ionic silver compounds, preferably silver nitrate with 60% percent silver content, into the spinning solution uniformly, the utilization efficiency of silver ions can be increased to 70%.

In contrast with the method of dissolving water-insoluble silver compound particles, this patent application provides a method of adding water soluble silver nitrate solution into the spinning solution. With this method the silver ions can be distributed evenly across the entire volume of polymer solution and therefore evenly to the fiber structure, providing a wound dressing having a prolonged and effective antimicrobial performance. The duration of antimicrobial effect of the wound dressing can be as long as 7 days.

In conclusion, this invention provides a method of uniformly distributing silver nitrate and silver ions into the fiber structure, and a method of manufacturing the antimicrobial fiber, fabric, and wound dressing with a high concentration of silver ions.

The present invention provides a wound dressing prepared by mixing water soluble silver nitrate with a spinning polymer solution, allowing silver ions to be distributed evenly across the entire fiber structure, enabling a more durable and faster release of silver ions during contact of the wound dressing with water, and providing a long-lasting antimicrobial efficiency, i.e., 7 days.

The objective of this invention is to provide a silver antimicrobial fiber, fabric, and wound dressing. The material is produced by adding silver nitrate directly into the spinning polymer solution and extruding the resulted mixture into fibers through wet spinning process. The silver content of the silver fiber made from this invention, expressed as the percentage of the dry weight of the polymer, is 0.01-10%, preferably 0.1-7%, and more preferably 0.5-5%.

The polymer referred to in this invention is an alginate or a chitosan. The alginate can be a high Guluronic alginate, or a high Mannuronic alginate or a mixture of both, where the term “high Guluronic alginate” refers to an alginate fiber in which the weight ratio of guluronic acid is higher than that of mannuronic acid and the term “high Mannuronic alginate” refers to an alginate fiber in which the weight ratio of mannuronic acid is higher than that of guluronic acid. The alginate fiber can be calcium alginate fiber or sodium/calcium alginate fiber. The chitosan fiber shall have a degree of deacetylation of at least 80%. The chitosan fiber can also be chemically modified, such as by carboxymethylation or acylation process, in order to improve its gelling and absorbency. The alginate or chitosan fibers shall have a fiber linear density of 1 to 5 dtex and a fiber length of 5 to 125 mm.

The wound dressing is made through a needle punching nonwoven process, a chemical bonding nonwovens process, or a weaving or knitting process. The fiber can be slightly longer, e.g. 30-100 mm, if a needle punching nonwoven process is used. The fiber can be slightly shorter, e.g. 3-15 mm, if a chemical bonding nonwoven process is used. Accordingly, the fiber length can be 20-85 mm if a weaving or knitting process is employed. When the silver wound dressing is made through the needle punching nonwoven process, its absorbency for a solution A (a solution containing 8.298 g/L of sodium chloride and 0.368 g/L of calcium chloride dehydrate in distilled water) measured according to Standard No. BS EN 13726-1, i.e., the total absorptive capacity in the presence of excess test liquid and in the absence of any applied load, is 1200% or above; and its dressing wet strengths in machine direction (MD) and in cross machine direction (CD) are 0.3 N/cm or above and 0.4 N/cm or above, respectively.

The second objective of this invention is to provide a method of manufacturing silver fiber and the silver wound dressing, which comprises the following steps:
a) dissolving silver nitrate in water;
b) adding a polymer, e.g. sodium alginate or chitosan, to the above silver solution to obtain a silver-containing polymer spinning solution. The ratio between the weight of silver ions and the dry weight of the polymer is between 0.01-10%, preferably 0.1-0.7%, and more preferably 0.5-5%;
c) extruding the above spinning solution into silver fibers through a respective wet spinning process;
d) converting the silver fibers into a fabric through a needle punching nonwoven process, a chemical bonding nonwovens process, a weaving process, or a knitting process; and
e) cutting, packing, and sterilizing the yielded fabric to obtain the silver wound dressing.

Steps a)-b) are conducted in the substantial absence of light so at to prevent photochemical reduction of the silver ions to elemental silver. Specifically, the mixing operations in steps a)-b) are conducted under a weak light (either daylight or artificial light) for a short period such that the solutions to be mixed does not undergo color changes. The rest operations in steps a)-b) including stirring and degassing are conducted in the absence of light.

Preferably, sodium hypochlorite can be added to the silver nitrate solution between steps a) and b). The weight of the sodium hypochlorite added shall be 0.005-2% of the weight of the polymer. Alternatively, sodium chloride can be added to the silver nitrate solution between steps a) and b). The weight of the sodium chloride added shall be 0.001%-11% of the weight of the polymer.

This invention also provides another method of manufacturing the antimicrobial wound dressing, which comprises the following steps:
a) dissolving silver nitrate in water;
b) adding sodium hypochlorite to the silver nitrate solution. The weight of the sodium hypochlorite added shall be 0.005-2% of the weight of the polymer used;
c) adding the polymer, e.g. sodium alginate or chitosan, to the above silver solution to obtain a silver-containing polymer spinning solution. The ratio between the weight of silver ions and the dry weight of the polymer is between 0.01-10%;
d) extruding the above spinning solution into silver fibers through respective wet spinning process;
e) converting the silver fibers to a fabric through a needle punching nonwoven process, a chemical bonding nonwovens process, a weaving process, or a knitting process; and
f) cutting, packing, and sterilizing the fabric to obtain the silver wound dressing.

Steps a)-c) are conducted in the substantial absence of light. Specifically, the mixing operations in steps a)-c) are conducted under a weak light (either daylight or artificial light) for a short period such that the solutions to be mixed does not undergo color changes. The rest operations in steps a)-c) including stirring and degassing are conducted in the absence of light.

Alternatively, this invention provides another method of manufacturing the antimicrobial wound dressing which comprises the following steps:
a) dissolving silver nitrate in water;
b) adding sodium chloride to the silver nitrate solution. The weight of the sodium chloride added shall be 0.001-11% of the weight of the polymer used;
c) adding the polymer, e.g. sodium alginate or chitosan, to the above silver solution to obtain a silver-containing polymer spinning solution. The ratio between the weight of silver ions and the dry weight of the polymer is between 0.01-10%;
d) extruding the above spinning solution into silver fibers through a respective wet spinning process;
e) converting the silver fibers to fabric through a needle punching nonwoven process, a chemical bonding nonwovens process, a weaving process, or a knitting process; and
f) cutting, packing, and sterilizing the fabric to obtain the silver wound dressing.

Steps a)-c) are conducted in the substantial absence of light. Specifically, the mixing operations in steps a)-c) are conducted under a weak light (either daylight or artificial light) for a short period such that the solutions to be mixed does not undergo color changes. The rest operations in steps a)-c) including stirring and degassing are conducted in the absence of light.

In an embodiment of this invention, the silver nitrate is mixed in water before adding the polymer material into the mix, which ensures that the silver nitrate be fully dissolved and mixed in water and then be distributed uniformly into the entire polymer solution. When the solution is extruded into fiber, and made into the wound dressing, the silver ions are also uniformly distributed in the structure of the fiber and the dressing. When the wound dressing is in contact with water or wound fluid, the external surface of the fiber/dressing is moisturized and releases silver ions first. When the water or wound fluid is further absorbed into the fiber/dressing structure, the silver ions in the inner structure of fiber/dressing can be released, thus allowing a continuing and long-lasting release of silver ions.

In an embodiment of the manufacturing method, a pre-mix of the polymer material in water is involved in the polymer mixing stage. At the start of the mixing, a small quantity of the polymer is mixed in water to achieve a solution viscosity of 200-1000 cps. Then, while the solution is stirred continuously, silver nitrate is added to the mix. Preferably more polymer can be added to the solution so that an ideal viscosity of the mixed solution of 500-1000 cps can be achieved. This viscosity can ensure a full mixing of silver nitrate without grouping or aggregation of the silver material. Then the remaining polymer is added to the mix while the solution is being stirred continuously. Mixing is kept for 20-90 mins, followed by the steps of degassing and extrusion to manufacture the silver antimicrobial fibers. This invention provides a method of manufacturing silver fiber by dissolving silver nitrate directly into the polymer solutions (such as alginate and chitosan) for wet spinning process without using reduction, stabilizing or dispersion agents in any steps of mixing or extrusion. The method is easy to use and is of low cost.

As the silver ions are evenly distributed in the fibers and the wound dressing, the wound dressing of this invention can provide continuous and long-lasting release of silver ions. Thus, the wound dressing is ideal for the management of chronic wounds and can be used to prevent or reduce wound infections.

### BRIEF DESCRIPTION OF THE DRAWINGS

FIG. 1 is a photograph of zone of inhibition of the dressing containing 0.5 wt. % silver for Staphylococcus aureus after 1 day;

FIG. 2 is a photograph of zone of inhibition of the dressing containing 0.5 wt. % silver for Staphylococcus aureus after 5 days;

FIG. 3 is a photograph of zone of inhibition of the dressing containing 0.5 wt. % silver for Staphylococcus aureus after 7 days;

FIG. 4 is a photograph of zone of inhibition of the dressing containing 1 wt. % silver for Escherichia coli after 1 day;

FIG. 5 is a photograph of zone of inhibition of the dressing containing 1 wt. % silver for Escherichia coli after 5 days;

FIG. 6 is a photograph of zone of inhibition of the dressing containing 1 wt. % silver for Escherichia coli after 7 days;

FIG. 7 is a photograph of zone of inhibition of the dressing containing 10 wt. % silver for Bacillus subtilis after 1 day;

FIG. 8 is a photograph of zone of inhibition of the dressing containing 10 wt. % silver for Bacillus subtilis after 7 days;

FIG. 9 is a photograph of zone of inhibition of the dressing containing 0.05 wt. % silver for Staphylococcus aureus after 1 day;

FIG. 10 is a photograph of zone of inhibition of the dressing containing 0.05 wt. % silver for Staphylococcus aureus after 7 days;

FIG. 11 is a photograph of zone of inhibition of the dressing containing 0.01 wt. % silver for Staphylococcus aureus after 1 day;

FIG. 12 is a photograph of zone of inhibition of the dressing containing 0.01 wt. % silver for Staphylococcus aureus after 7 days; and

FIG. 13 shows a silver releasing profile of the dressing made from Example 1 in 10 ml of simulated wound exudate.

### DETAILED DESCRIPTION

This invention is further illustrated through the following examples and figures.

In the following examples, the operations of preparing, mixing, and degassing the polymer and silver nitrate solutions were conducted in a vessel with a cover, such as a stainless-steel tank, in the substantial absence of light.

The calculation for mixing and components weights is summarized as follows:

When the dry weight of sodium alginate powder is 6 kg and the moisture content of the material is 11%, the weight of sodium alginate at ambient conditions is 6 kg÷(1-11%)=6.74 kg. When preparing a polymer solution of sodium alginate at 5% solid content, the quantity of water needed for the mixing is 6 kg÷5%×95%=114 kg.

The silver content in silver nitrate is 60%. To make silver alginate fibers with 0.5% silver content, the weight of silver in 6 kg of sodium alginate powder is calculated as: 6 kg×0.5%=0.03 kg, which requires 0.03 kg÷60%=0.05 kg of silver nitrate.

### Example 1

The manufacturing method for antimicrobial fibers and wound dressing containing 0.5% by weight silver:

To make 6 kg of silver alginate fibers with 0.5% silver content, it needed 50 g of silver nitrate, 6.74 kg of sodium alginate, and 114 L of water.

114 L of water was added to a mixing vessel.

50 g of silver nitrate was added to the mixing vessel. The mixer was started to fully dissolve and mix the silver nitrate in the water, and then while the mixer was kept running, 1 kg of sodium alginate powder was added to the solution. After that, the mixer was kept running and the mixture of undissolved alginate and silver nitrate was checked to ensure that the viscosity reach the pre-stated ideal level.

The remaining sodium alginate was added to the solution while the mixer was kept running.

After the sodium alginate was completely dispersed, the solution was kept still for 24 hours in a sealed stainless-steel vessel for natural degassing. Because of the high viscosity of the polymer solution, the silver ion was evenly distributed in the alginate polymer solution.

After the degassing was completed, the polymer solution was ready to be extruded to calcium silver alginate fiber through a standard wet-spinning process. Typically, silver-containing sodium alginate solution was pumped through a spinneret into a coagulating bath to convert the sodium alginate to calcium alginate fiber, followed with stretching bath, washing, drying, crimping, and cutting.

This process yielded white or off-white fibers with 0.5 wt. % silver content.

The silver fibers were converted into nonwoven felt and cut into 10×10 cm pads for packaging. The dressing was irradiated by 25-40 kGy of gamma ray.

The silver alginate dressing with 0.5% silver content was obtained.

### Example 2

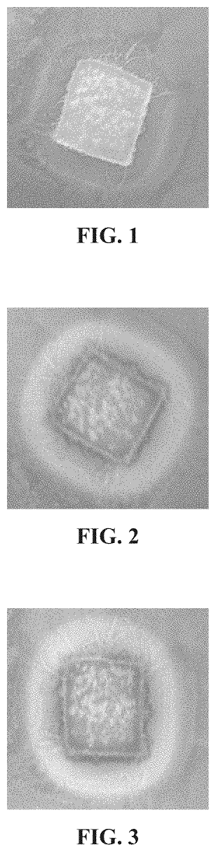

The dressing from Example 1 was cut into 2×2 cm, wetted, and then placed into a petri dish that was covered evenly with Staphylococcus aureus. The petri dish was placed into a 37° C. incubator for 7 days, and the growth of the microorganic was observed. When the silver ions were released from the dressing, the microorganisms surrounding the dressing sample were killed, creating a visible zone of inhibition. The larger the zone is, the better the antimicrobial property the dressing has. FIG. 1 displays a zone of inhibition of the dressing containing 0.5 wt. % silver for Staphylococcus aureus after 1 day; FIG. 2 displays a zone of inhibition of the dressing containing 0.5 wt. % silver for Staphylococcus aureus after 5 days; FIG. 3 displays a zone of inhibition of the dressing containing 0.5 wt. % silver for Staphylococcus aureus after 7 days. It can be seen that the dressing with 0.5% silver content still has a good antimicrobial function after 7 days.

### Example 3

The manufacturing method for antimicrobial fibers and wound dressing containing 1% by weight silver:

To make 6 kg of silver alginate fibers with 1% silver content, it needed 100 g of silver nitrate, 6.74 kg of sodium alginate, and 114 L of water.

114 L of water was added to a mixing vessel.

100 g of silver nitrate was added to the mixing vessel. The mixer was started to fully dissolve and mix the silver nitrate in the water, and then while the mixer was kept running, 1 kg of sodium alginate powder was added to the solution. After that, the mixer was kept running, and the mixture of undissolved alginate and silver nitrate was checked to ensure that the viscosity reach the pre-stated ideal level.

The remaining sodium alginate was added to the solution while the mixer was kept running.

After the sodium alginate was completely dispersed, the solution was kept still for 24 hours for natural degassing. Because of the high viscosity of the polymer solution, the silver ion was evenly distributed in the alginate polymer solution.

After the degassing was completed, the polymer solution was ready to be extruded to calcium silver alginate fiber through a standard wet-spinning process. Typically, the silver-containing sodium alginate solution was pumped through a spinneret into a coagulating bath to convert the sodium alginate to calcium alginate fiber, followed with stretching bath, washing, drying, crimping, and cutting.

This process yielded white or off-white fibers with 1 wt. % silver content.

The silver fibers were converted into nonwoven felt and cut into 10×10 cm pads for package. The dressing was irradiated by 25-40 kGy of gamma ray.

The silver alginate dressing with 1% silver content was obtained.

### Example 4

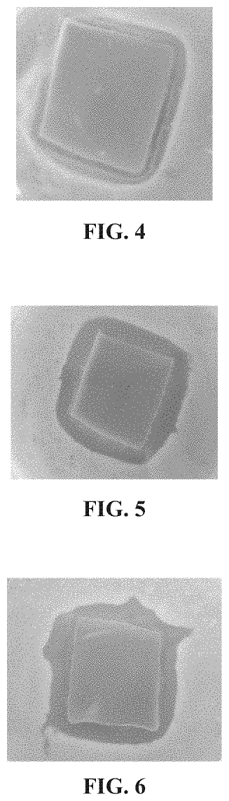

The dressing from Example 3 was cut into 2×2 cm, wetted, and placed into a petri dish that was covered evenly with Escherichia coli. The petri dish was placed into a 37° C. incubator for 7 days, and the growth of the microorganic was observed. When the silver ions were released from the dressing, the microorganisms surrounding the dressing sample were killed, creating a visible zone of inhibition. FIG. 4 displays a zone of inhibition of the dressing containing 1 wt. % silver for Escherichia coli after 1 day; FIG. 5 displays a zone of inhibition of the dressing containing 1 wt. % silver for Escherichia coli after 5 days; FIG. 6 displays a zone of inhibition of the dressing containing 1 wt. % silver for Escherichia coli after 7 days. It can be seen that the dressing with 1% silver content has a good antimicrobial function after 7 days.

### Example 5

The manufacturing method for antimicrobial fibers and wound dressing containing 10% by weight silver:

To make 6 kg of silver alginate fibers with 10% silver content, it needed 1000 g of silver nitrate, 6.74 kg of sodium alginate, and 114 L of water.

114 L of water was added to a mixing vessel.

1000 g of silver nitrate was added to the mixing vessel. The mixer was started to fully dissolve and mix the silver nitrate in the water, and then while the mixer was kept running, 1 kg of sodium alginate powder was added to the solution. After that, the mixer was kept running, and the mixture of undissolved alginate and silver nitrate was checked to ensure that the viscosity reach the ideal level to prevent re-grouping of the silver material.

The remaining sodium alginate was added to the solution while the mixer was kept running.

After the sodium alginate was completely dispersed, the solution was kept still for 24 hours for natural degassing. Because of the high viscosity of the polymer solution, the silver ion was kept suspended and evenly distributed in the alginate polymer solution.

After the degassing was completed, the polymer solution was ready to be extruded to calcium silver alginate fiber through a standard wet-spinning process. Typically, the silver-containing sodium alginate solution was pumped through a spinneret into a coagulating bath to convert the sodium alginate to calcium alginate fiber, followed with stretching bath, washing, drying, crimping, and cutting.

This process yielded white or off-white fibers with 10 wt. % silver content.

The silver fibers were converted into nonwoven felt and cut into 10×10 cm pads for package. The dressing was irradiated by 25-40 kGy of gamma ray.

The silver alginate dressing with 10% silver content was obtained.

### Example 6

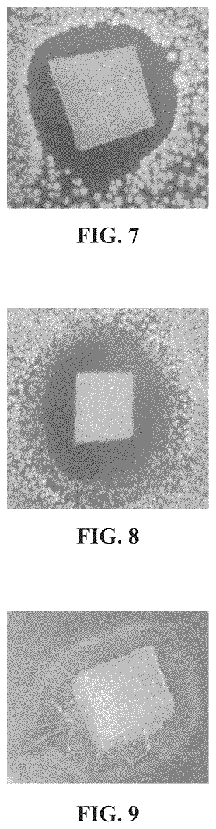

The dressing from Example 5 was cut into 2×2 cm, wetted, and then placed into a petri dish that was covered evenly with Staphylococcus aureus. The petri dish was placed into a 37° C. incubator for 7 days, and the growth of the microorganic was observed. When the silver ions were released from the dressing, the microorganisms surrounding the dressing sample were killed, creating a visible zone of inhibition. FIG. 7 displays a zone of inhibition for Staphylococcus aureus after 1 day; FIG. 8 displays a zone of inhibition after 5 days; FIG. 9 displays a zone of inhibition after 7 days. It can be seen that the dressing produces an excellent zone of inhibition in 7 days.

### Example 7

The manufacturing method for antimicrobial fibers and wound dressing containing 0.05% by weight silver:

To make 6 kg of silver alginate fibers with 0.05% silver content, it needed 5 g of silver nitrate, 6.74 kg of sodium alginate and 114 L of water.

114 L of water was added to a mixing vessel.

5 g of silver nitrate was added to the mixing vessel. The mixer was started to fully dissolve and mix the silver nitrate in the water.

6.74 kg of sodium alginate was added to the solution.

After the sodium alginate was completely dispersed, the solution was kept still for 24 hours for natural degassing. Because of the high viscosity of the polymer solution, the silver ion was evenly distributed in the alginate polymer solution.

After the degassing was completed, the polymer solution was ready to be extruded to calcium silver alginate fiber through a standard wet-spinning process.

This process yielded white or off-white fibers with 0.05% (by weight) silver content.

The silver fibers were converted into nonwoven felt and cut into 10×10 cm pads for package. The dressing was irradiated by 25-40 kGy of gamma ray.

The silver alginate dressing with 0.05% silver content was obtained.

### Example 8

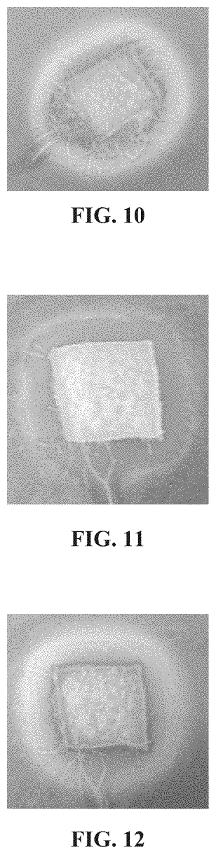

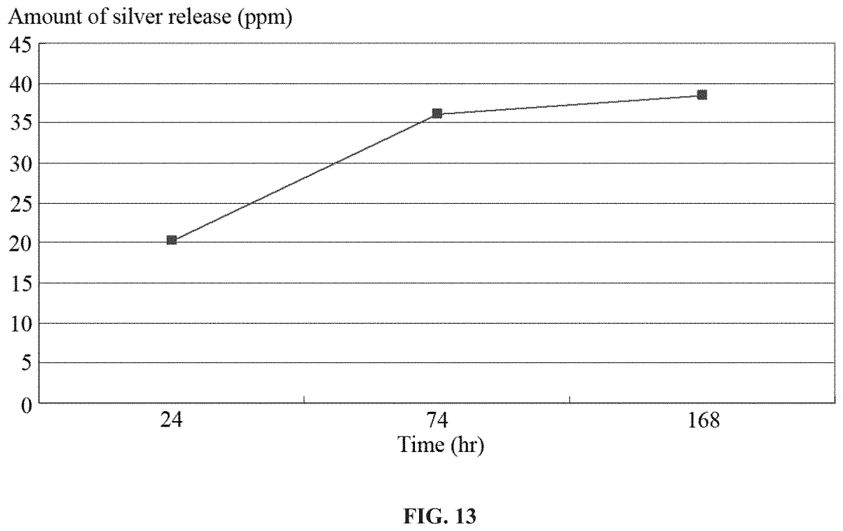

The dressing from Example 7 was cut into 2×2 cm, wetted, and then placed into a petri dish that was covered evenly with Staphylococcus aureus. The petri dish was placed into a 37° C. incubator for 7 days, and the growth of the microorganic was observed. When the silver ions were released from the dressing, the microorganisms surrounding the dressing sample were killed, creating a visible zone of inhibition. FIG. 9 displays a zone of inhibition of dressing with 0.05% silver for Staphylococcus aureus after 1 day; FIG. 10 displays a zone of inhibition after 7 days. These indicate that the dressing with 0.05% of silver still has a good antimicrobial property.

### Example 9

The manufacturing method for antimicrobial fibers and wound dressing containing 0.01% by weight silver:

To make 6 kg of silver alginate fibers with 0.01% silver content, it needed 1 g of silver nitrate, 6.74 kg of sodium alginate and 114 L of water.

114 L of water was added to the mixing vessel.

All of the silver nitrate was added to the mixing vessel. The mixer was started to fully dissolve and mix the silver nitrate in the water.4. All of the sodium alginate was added to the solution.

After the sodium alginate was completely dispersed, the solution was kept still for 24 hours for natural degassing. Because of the high viscosity of the polymer solution, the silver ion was evenly distributed in the alginate polymer solution.

After the degassing was completed, the polymer solution was ready to be extruded to calcium silver alginate fiber through a standard wet-spinning process.

This process yielded white or off-white fibers with 0.01% (by weight) silver content.

The silver fibers were converted into nonwoven felt and cut into 10×10 cm pads for package. The dressing was irradiated by 25-40 kGy of gamma ray.

The silver alginate dressing with 0.01% silver content was obtained.

### Example 10

The dressing from Example 9 was cut into 2×2 cm, wetted, and placed into a petri dish that was covered evenly with Staphylococcus aureus. The petri dish was placed into a 37° C. incubator for 7 days, and the growth of the microorganic was observed. When the silver ions are released from dressing, the microorganisms surrounding the dressing sample were killed, creating a visible zone of inhibition. FIG. 11 displays a zone of inhibition of dressing with 0.01% silver content for Staphylococcus aureus after 1 day; FIG. 12 displays a zone of inhibition after 7 days. These figures indicate that the dressing with 0.01% of silver still has some antimicrobial property.

### Example 11

The manufacturing method for antimicrobial chitosan fibers and chitosan wound dressing containing 1.1% by weight silver:

Target silver content was 1.1%, quantity of chitosan powder or flakes was 200 g, the moisture content of the chitosan was 10% by weight. At 5 wt. % solid content, 3420 ml of 2 wt. % acetic acid solution was needed. The dry weight of the chitosan powder was 180 g.

To make 180 g of chitosan fiber with target silver content of 1.1%, 3.3 g of silver nitrate was required.

All of the silver nitrate was added to a small container that had been pre-charged with the required amount of acetic acid solution. The mixer was started to dissolve the silver nitrate.

30 g of chitosan powder was added to the acetic acid solution prepared in the above step.

When the chitosan powder was fully dissolved and the solution reached the ideal viscosity, the remaining powder was added.

When all the chitosan was fully mixed into the solution, the mixer was removed, and the solution was kept still for 24 hours for natural degassing.

After the degassing was completed, the polymer solution was ready to be extruded into silver chitosan fiber through a standard wet-spinning process. Typically, silver-containing chitosan solution was pumped through a spinneret into a bath of 5 wt. % sodium hydrate solution to convert the chitosan solution into filaments, followed with stretching bath, washing, drying, crimping, and cutting.

This process yielded white or creamy colored fibers with 1.1% (by weight) silver content.

The silver fibers were converted into nonwoven felt on standard textile machine, and cut into 10×10 cm pads for package. The dressing was irradiated by 25-40 kGy of gamma ray.

The silver chitosan dressing with 1.1% silver content was obtained.

### Example 12

The manufacturing method for antimicrobial fibers and wound dressing containing silver chloride:

114 L of water was added to the mixing vessel.

5 g of silver nitrate was added to the mixing vessel. The mixer was started to fully dissolve and mix the silver nitrate in the water. 1.72 g of sodium chloride was added to the solution while the mixer was kept running. The weight of sodium chloride was at a molar ratio of 1:1 to silver nitrate. This converted the silver nitrate to silver chloride. Another 1 kg of sodium alginate was added to the mix while the mixer was kept running.

The remaining 5.74 kg of sodium alginate was added to the solution while the mixer was on.

After the sodium alginate was completely dispersed, the solution was kept still for 24 hours for natural degassing. Because of the high viscosity of the polymer solution, the silver chloride was evenly mixed with the alginate polymer solution without aggregation of silver chloride.

After the degassing was completed, the polymer solution was ready to be extruded into silver alginate fiber through a standard wet-spinning process, i.e. metering pump, coagulant bath, stretching, washing, drying, crimping, and cutting.

This process yielded white or off-white silver alginate fibers with 0.05% (by weight) silver content.

The silver fibers were converted into nonwoven felt and cut into 10×10 cm pads for package. The dressing was irradiated by 25-40 kGy of gamma ray.

The silver alginate dressing with 0.05% silver content was obtained.

### Example 13

The manufacturing method for antimicrobial fibers and wound dressing containing silver hypochlorite:

114 L of water was added to the mixing vessel.

5 g of silver nitrate was added to the mixing vessel. The mixer was started to fully dissolve and mix the silver nitrate in the water. 2.19 g of sodium hypochlorite was added to the solution while the mixer was kept running. The weight of sodium hypochlorite was at a molar ratio of 1:1 to silver nitrate. This converted the silver nitrate into silver hypochlorite. Another 1 kg of sodium alginate was added to the mix while the mixer was kept running.

The remaining 5.74 kg of sodium alginate was added to the solution while the mixer was on.

After the sodium alginate was completely dispersed, the solution was kept still for 24 hours for natural degassing. Because of the high viscosity of the polymer solution, the silver hypochlorite was evenly mixed with the alginate polymer solution without aggregation of silver chloride.

After the degassing was completed, the polymer solution was ready to be extruded into silver alginate fibers through a standard wet-spinning process, i.e. metering pump, coagulant bath, stretching, washing, drying, crimping, and cutting.

This process yielded white or off-white silver alginate fibers with 0.05% (by weight) silver content.

The silver fibers were converted into nonwoven felt and cut into 10×10 cm pads for package. The dressing was irradiated by 25-40 kGy of gamma ray.

The silver alginate dressing with 0.05% silver content was obtained.

### Example 14

The manufacturing method for antimicrobial fibers and wound dressing containing silver chloride:

114 L of water was added to the mixing vessel.

100 g of silver nitrate was added to the mixing vessel. The mixer was started to fully dissolve and mix the silver nitrate in the water. 34.4 g of sodium chloride was added to the solution while mixer was kept running. The amount of sodium chloride added was at a molar ratio of 1:1 to silver nitrate. This converted the silver nitrate to silver chloride. Another 1 kg of sodium alginate was added to the mix while the mixer was kept running.

The remaining 5.74 kg of sodium alginate was added to the solution while the mixer was on.

After the sodium alginate was completely dispersed, the solution was left still for 24 hours for natural degassing. Because of the high viscosity of the polymer solution, the silver chloride was evenly mixed with the alginate polymer solution without aggregation.

After the degassing was completed, the polymer solution was ready to be extruded into silver alginate fiber through a standard wet-spinning process, i.e. metering pump, coagulant bath, stretching, washing, drying, crimping, and cutting.

This process yielded white or off-white silver alginate fibers with 1.0% (by weight) silver content.

The silver fibers were converted into nonwoven felt and cut into 10×10 cm pads for package. The dressing was irradiated by 25-40 kGy of gamma ray.

The silver alginate dressing with 1.0% silver content was obtained.

### Example 15

The manufacturing method for antimicrobial fibers and wound dressing containing silver hypochlorite:

114 L of water was added to the mixing vessel.

100 g of silver nitrate was added to the mixing vessel. The mixer was started to fully dissolve and mix the silver nitrate in the water. 43.8 g of sodium hypochlorite was added to the solution while the mixer was kept running. The amount of sodium hypochlorite was at a molar ratio of 1:1 to silver nitrate. This converted the silver nitrate into silver hypochlorite. Another 1 kg of sodium alginate was added to the mix while the mixer was kept running.

The remaining 5.74 kg of sodium alginate was added to the solution while the mixer was on.

After the sodium alginate was completely dispersed, the solution was left still for 24 hours for natural degassing. Because of the high viscosity of the polymer solution, the silver hypochlorite was evenly mixed with the alginate polymer solution without aggregation.

After the degassing was completed, the polymer solution was ready to be extruded into silver alginate fibers through a standard wet-spinning process, i.e. metering pump, coagulant bath, stretching, washing, drying, crimping, and cutting.

This process yielded white or off-white silver alginate fibers with 1% (by weight) silver content.

The silver fibers were converted into nonwoven felt and cut into 10×10 cm pads for package. The dressing was irradiated by 25-40 kGy of gamma ray.

The silver alginate dressing with 1% silver content was obtained.

### Example 16

The manufacturing method for antimicrobial fibers and wound dressing containing silver chloride:

114 L of water was added to the mixing vessel.

1000 g of silver nitrate was added to the mixing vessel. The mixer was started to fully dissolve and mix the silver nitrate in the water. 344 g of sodium chloride was added to the solution while the mixer was kept running. The amount of sodium chloride added was at a molar ratio of 1:1 to silver nitrate. This converted the silver nitrate to silver chloride. Another 1 kg of sodium alginate was added to the mix while the mixer was kept running.

The remaining 5.74 kg of sodium alginate was added to the solution while the mixer was on.

After the sodium alginate was completely dispersed, the solution was left still for 24 hours for natural degassing. Because of the high viscosity of the polymer solution, the silver chloride was evenly mixed with the alginate polymer solution without aggregation.

After the degassing was completed, the polymer solution was ready to be extruded into silver alginate fiber through a standard wet-spinning process, i.e. metering pump, coagulant bath, stretching, washing, drying, crimping, and cutting.

This process yielded white or off-white silver alginate fibers with 10% (by weight) silver content.

The silver fibers were converted into nonwoven felt and cut into 10×10 cm pads for package. The dressing was irradiated by 25-40 kGy of gamma ray.

The silver alginate dressing with 10% silver content was obtained.

### Example 17

The manufacturing method for antimicrobial fibers and wound dressing containing silver hypochlorite:

114 L of water was added to the mixing vessel.

1000 g of silver nitrate was added to the mixing vessel. The mixer was started to fully dissolve and mix the silver nitrate in the water. 438 g of sodium hypochlorite was added to the solution while the mixer was kept running. The amount of sodium hypochlorite was at a molar ratio of 1:1 to silver nitrate. This converted the silver nitrate into silver hypochlorite. Another 1 kg of sodium alginate was added to the mix while the mixer was kept running.

The remaining 5.74 kg of sodium alginate was added to the solution while the mixer was on.

After the sodium alginate was completely dispersed, the solution was left still for 24 hours for natural degassing. Because of the high viscosity of the polymer solution, the silver hypochlorite was evenly mixed with the alginate polymer solution without aggregation.

After the degassing was completed, the polymer solution was ready to be extruded into silver alginate fibers through a standard wet-spinning process, i.e. metering pump, coagulant bath, stretching, washing, drying, crimping, and cutting.

This process yielded white or off-white silver alginate fibers with 10% (by weight) silver content.

The silver fibers were converted into nonwoven felt and cut into 10×10 cm pads for package. The dressing was irradiated by 25-40 kGy of gamma ray.

The silver alginate dressing with 10% silver content was obtained.

### Example 18

Determination of silver release:

In order to establish the silver release profile of the silver-containing antimicrobial wound dressing, the silver dressing from Example 1 was cut into 2.5×2.5 cm and placed into 10 ml of simulated wound fluid. The sample was incubated in a water bath at 37° C. and was kept shaking at 60-80 rpm for 7 days. The silver ions were released into the wound fluid and the amount of the silver in the solution was tested at the time points of 24 hrs, 72 hrs and 168 hrs. The following Table 1 gives the amount of silver released into 10 ml of simulated wound exudates at the relevant time points. It can be seen that the silver release increases with the time, with the maximum silver release of 38.4 ppm at the time point of 168 hrs.

TABLE 1
Silver release in 10 ml of
simulated wound exudate

| | Silver |
|---|---|
| Time | release |
| (hrs) | (ppm) |
| 24 | 20.3 |
| 72 | 36.1 |
| 168 | 38.4 |

The invention claimed is:

1. A wound dressing, comprising fibers, the fibers comprising silver ions; wherein the content of the silver ions in the fiber is between 0.01-10% by weight.

2. The wound dressing of claim 1, wherein the fiber further comprises chitosan fibers or alginate fibers.

3. The wound dressing of claim 2, wherein the alginate fibers are high guluronic alginate fiber, high mannuronic alginate fiber, or a mixture thereof.

4. The wound dressing of claim 2, wherein the alginate fibers are:
calcium alginate fibers or
calcium and sodium alginate fiber.

5. The wound dressing of claim 2, wherein the chitosan fiber has a degree of deacetylation of at least 80%.

6. The wound dressing of claim 2, wherein the chitosan fibers are carboxymethylated or acylated chitosan fibers.

7. The wound dressing of claim 1, wherein the fibers have a linear density of 1-5 dtex and a fiber length of 5-125 mm.

8. The wound dressing of claim 1, wherein the silver dressing is a needle punched nonwoven fabric having dressing wet strengths in machine direction (MD) and in cross machine direction (CD) of 0.3 N/cm or above and 0.4 N/cm or above, respectively.

9. A method of manufacturing the wound dressing of claim 1, the method comprising:
a) dissolving silver nitrate in water in the substantial absence of light thereby yielding a silver nitrate solution;
b) adding a polymer to the silver nitrate solution in the substantial absence of light thereby yielding a polymer solution comprising silver ions, wherein a ratio of the weight of silver ions to the dry weight of the polymer is 0.01-10%, and the polymer is chitosan or alginate;
c) extruding the polymer solution comprising silver ions into fibers comprising silver ions through a wet spinning process;
d) fabricating the fibers comprising silver ions into a nonwoven fabric through needle punching process or chemical bonding process; and
e) cutting, packing, and sterilizing the nonwoven fabric to obtain the wound dressing.

10. The method of claim 9, wherein sodium hypochlorite is added to the silver nitrate solution between step a) and step b) in the substantial absence of light, and the weight ratio of the sodium hypochlorite to the polymer is between 0.005% and 2%.

11. The method of claim 9, wherein sodium chloride is added to the silver nitrate solution between step a) and step b) in the substantial absence of light, and the weight ratio of the sodium chloride to the polymer is between 0.001% and 11.0%.

12. A method of manufacturing the wound dressing of claim 1, the method comprising:
a) dissolving a first portion of a polymer in water in the substantial absence of light thereby yielding a solution, wherein the solution has a viscosity of between 200-1000 cps, and the polymer is chitosan or alginate;
b) dissolving silver nitrate in the solution of a) in the substantial absence of light thereby yielding a mixture, and after stirring the mixture for 20-90 mins in the substantial absence of light, adding a second portion of the polymer to the mixture in the substantial absence of light, thereby yielding a polymer solution comprising silver ions;
c) degassing the polymer solution comprising silver ions in the substantial absence of light;
d) extruding the polymer solution comprising silver ions into fibers comprising silver ions through a wet spinning process;
e) fabricating the fibers comprising silver ions into a nonwoven fabric through needle punching process or chemical bonding process; and
f) cutting, packing, and sterilizing the nonwoven fabric to obtain the wet-spun fibrous silver wound dressing.
